(19) United States
(12) Patent Application Publication
(10) Pub. No.: US 2022/0351025 A1
(43) Pub. Date: Nov. 3, 2022

(54) DATA PROCESSING DEVICE AND DATA PROCESSING METHOD

(71) Applicant: CANON MEDICAL SYSTEMS CORPORATION, Tochigi (JP)

(72) Inventors: Hidenori TAKESHIMA, Kawasaki (JP); Hideaki KUTSUNA, Kawasaki (JP)

(73) Assignee: CANON MEDICAL SYSTEMS CORPORATION, Tochigi (JP)

(21) Appl. No.: 17/660,675

(22) Filed: Apr. 26, 2022

(30) Foreign Application Priority Data
Apr. 28, 2021 (JP) 2021-076825

(51) Int. Cl. G06N 3/04; G06N 3/063; G06N 3/08

(52) U.S. Cl. CPC G06N 3/0481; G06N 3/063; G06N 3/08; G16H 30/40

(57) ABSTRACT

A data processing device according to an embodiment includes a processing circuit. The processing circuit includes a complex number neural network with an activation function by which an output varies according to an argument of complex.

### CROSS-REFERENCE TO RELATED APPLICATIONS

This application is based upon and claims the benefit of priority from Japanese Patent Application No. 2021-076825, filed on Apr. 28, 2021; the entire contents of which are incorporated herein by reference.

### FIELD

Embodiments described herein relate generally to a data processing device and a data processing method.

### BACKGROUND

In machine learning using a neural network, a real number neural network is used as standard.

In a medical data processing apparatus, such as a magnetic resonance imaging apparatus or an ultrasound diagnosis apparatus, because a lot of signal processing using complex numbers is used, various applications are expected in using a complex number neural network.

### BRIEF DESCRIPTION OF THE DRAWINGS

FIG. 1 is a diagram illustrating an example of a data processing device according to an embodiment;

FIG. 2 is a diagram illustrating an example of a neural network according to the embodiment;

FIG. 3 is a diagram illustrating the neural network according to the embodiment;

FIG. 4 is a diagram illustrating an example of a configuration of the neural network according to the embodiment;

FIG. 5 is a diagram illustrating an example of the configuration of the neural network according to the embodiment;

FIG. 6 is a diagram illustrating an example of the configuration of the neural network according to the embodiment;

FIG. 7 is a diagram illustrating an example of the configuration of the neural network according to the embodiment;

FIG. 8 is a diagram illustrating an example of a magnetic resonance imaging apparatus according to the embodiment; and

FIG. 9 is a diagram illustrating an example of an ultrasound diagnosis apparatus according to the embodiment.

### DETAILED DESCRIPTION

A data processing device that is provided in one aspect of the present disclosure includes a processing circuit. The processing circuit includes a complex number neural network with an activation function by which an output varies according to an argument of complex.

### Embodiment

With reference to the accompanying drawings, an embodiment of a data processing device and a data processing method will be described in detail below.

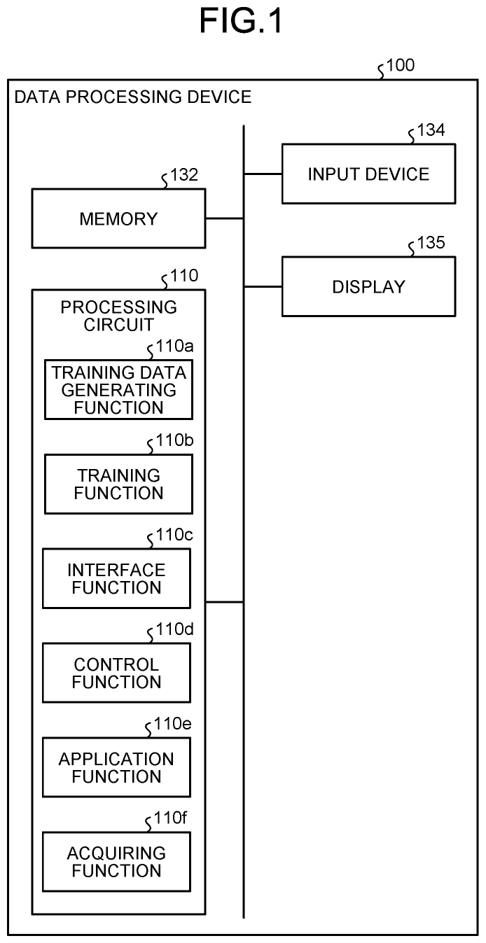

First of all, using FIG. 1, a configuration of a data processing device 100 according to the embodiment will be described.

The data processing device 100 is a device that generates data using machine learning. For example, the data processing device 100 executes processing of analysis signal data obtained by complexification of an actual signal utilizing orthogonality, generation of a trained model, execution of the trained model, etc.

The data processing device 100 includes a processing circuit 110, a memory 132, an input device 134, and a display 135. The processing circuit 110 includes a training data generating function 110a, a training function 110b, an interface function 110c, a control function 110d, an application function 110e, and an acquiring function 110f.

In the embodiment, each of processing functions enabled by the training data generating function 110a, the training function 110b, the interface function 110c, the control function 110d, the application function 110e, and the acquiring function 110f and a trained model (for example, a neural network) are stored in a form of computer-executable programs in the memory 132. The processing circuit 110 is a processor that reads the programs from the memory 132 and that executes the programs, thereby implementing the functions corresponding to the respective programs. In other words, the processing circuit 110 having read each of the programs has each of the functions illustrated in the processing circuit 110 in FIG. 1. The processing circuit 110 having read the program corresponding to the trained model (neural network) is able to perform a process according to the trained model. Note that, it is described using FIG. 1 that the functions of the processing circuit 110 are implemented by a single processing circuit; however, a plurality of independent processors may be combined to configure the processing circuit 110 and each of the processors may execute the program, thereby implementing the function. In other words, the above-described functions may be configured as programs and a processing circuit may execute each program. A single processing circuit may implement at least two of the functions that the processing circuit 110 has. In another example, a specific function may be installed in a dedicated independent program execution circuit.

Note that the processing circuit 110, the training data generating function 110a, the training function 110b, the interface function 110c, the control function 110d, the application function 110e, and an acquiring function 110f are respectively examples of a processor, a generator, an input unit (training unit), a receiving unit, a controller, an application unit, and an acquiring unit.

The term “processor” used in the description above means, for example, a central processing unit (CPU), a GPU graphical processing unit (GPU), or a circuit, such as an application specific integrated circuit (ASIC) or a programmable logic device (for example, a simple programmable logic device (SPLD), a complex programmable logic device (CPLD), or a field programmable gate array (FPGA)). The processor reads the programs that are saved in the memory 132 and executes the programs, thereby implementing the functions.

A configuration in which, instead of saving the programs in the memory 132, the programs may be embedded directly in the circuit of the processor. In this case, the processor reads the programs embedded in the circuit and executes the read programs, thereby implementing the functions. Thus, for example, instead of saving the trained model in the memory 132, a program relating to the trained model may be directly embedded in the circuit of the processor.

By the training data generating function 110a, the processing circuit 110 generates training data for training based on data, a signal, and an image that are acquired by the interface function 110c.

By the training function 110b, the processing circuit 110 performs training using the training data that is generated by the training data generating function 110a and generates a trained model.

By the interface function 110c, the processing circuit 110 acquires the data, the signal, the image, etc., for signal generation by the application function 110e from the memory 132.

By the control function 110d, the processing circuit 110 controls entire processes performed by the data processing device 100. Specifically, by the control function 110d, the processing circuit 110 controls the processes performed by the processing circuit 110 based on various setting requests that are input from an operator via the input device 134 and various control programs and various types of data that are read from the memory 132.

By the application function 110e, the processing circuit 110 generates a signal based on a result of the process performed using the training data generation function 110a and the training function 110b. By the application function 110e, the processing circuit 110 applies the trained model that is generated by the training function 110b to an input signal and generates a signal based on the result of application of the trained model.

The memory 132 consists of a semiconductor memory device, such as a random access memory (RAM) or a flash memory, a hard disk, an optical disk, or the like. The memory 132 is a memory that stores data, such as signal data for display that is generated by the processing circuit 110 or signal data for training.

The memory 132 stores various types of data, such as a control program for signal processing and display processing, as required.

The input device 134 receives various instructions and information inputs from the operator. The input device 134 is, for example, a pointing device, such as a mouse or a track ball, a selective device, such as a mode switching switch, or an input device, such as a keyboard.

Under the control of the control function 110d, etc., the display 135 displays a graphical user interface (GUI) for receiving an input of an imaging condition, a signal that is generated by the control function 110d, or the like, etc. The display 135 is, for example, a display device, such as a liquid crystal display device. The display 135 is an example of a display unit. The display 135 includes a mouse, a keyboard, a button, a panel switch, a touch command screen, a foot switch, a trackball, a joystick, etc.

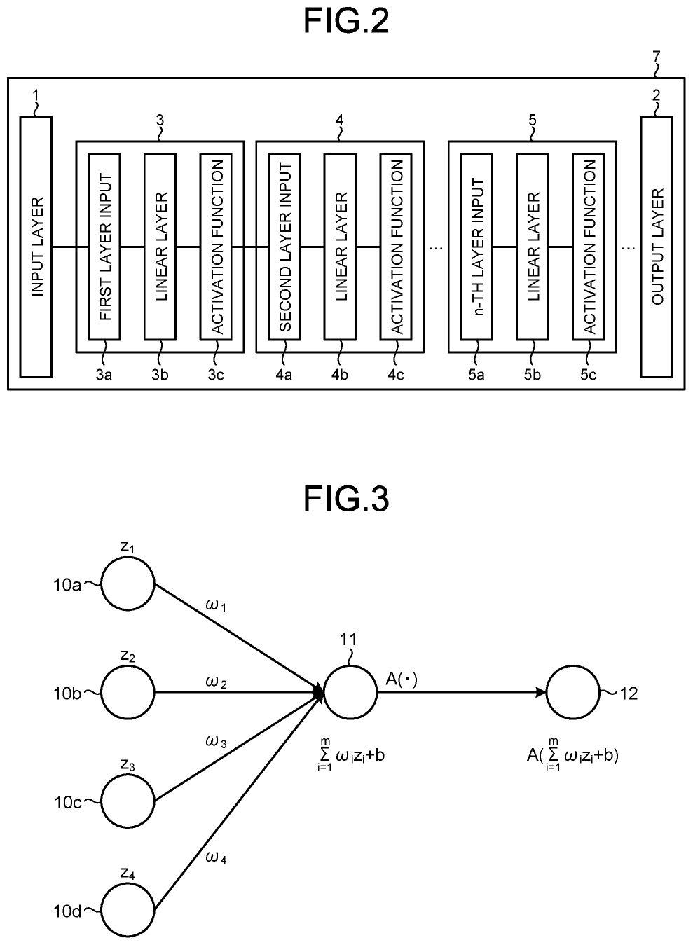

Using FIGS. 2 to 4, a configuration of a neural network according to the embodiment will be described.

FIG. 2 illustrates an example of mutual connection among layers in a neural network 7 that is used for machine learning by the processing circuit 110 including the training function 110b. The neural network 7 consists of an input layer 1, an output layer 2, and intermediate layers 3, 4 and 5 between the input layer 1 and the output layer 2. Each of the intermediate layers consists of a layer relating to input (referred to as an input layer in each of the layers), a linear layer, and a layer relating to a process using an activation function (referred to as an activation layer below). For example, the intermediate layer 3 consists of an input layer 3a, a linear layer 3b and an activation layer 3c, the intermediate layer 4 consists of an input layer 4a, a linear layer 4b and an activation layer 4c, and the intermediate layer 5 consists of an input layer 5a, a linear layer 5b and an activation layer 5c. Each of the layers consists of a plurality of nodes (neurons).

The data processing device 100 according to the embodiment applies a linear layer of complex number coefficient and non-linear activation (an activation function) to medical data having a complex number value. In other words, by the training function 110b, the processing circuit 110 trains the neural network 7 that applies a complex number coefficient linear layer and non-linear activation (an activation function) to medical data that has a complex number value, thereby generating a trained model. The data processing circuit 100 stores the generated trained model, for example, in the memory 132 as required.

Note that the data that is input to the input layer 1 is, for example, complex number data obtained by performing discrete sampling on an electric signal by quadrature detection.

Data that is output from the output layer 2 is, for example, complex number data from which noise has been removed.

When the neural network 7 according to the embodiment is a convolutional neural network (CNN), the data that is input to the input layer 1 is, for example, data represented by two-dimensional array whose size is 32×32, or the like, and the data that is output from the output layer 2 is, for example, data represented by two-dimensional array whose size is 32×32, or the like. The size of the data that is input to the input layer 1 and the size of the data that is output from the output layer 2 may be equal to or different from each other. Similarly, the number of nodes in the intermediate layer may be equal to the number of nodes of the layer preceding or following the intermediate layer.

Subsequently, generation of a trained model according to the embodiment, that is, a training step will be described. By the training function 110b, the processing circuit 110 performs, for example, machine learning on the neural network 7, thereby generating a trained model. Performing machine learning here means that weights in the neural network 7 consisting of the input layer 1, the intermediate layer 3, 4 and 5 and the output layer 2 are determined, specifically, a set of coefficients characterizing coupling between the input layer 1 and the intermediate layer 3, a set of coefficients characterizing coupling between the intermediate layer 3 and the intermediate layer 4, and a set of coefficients characterizing coupling between the intermediate layer 5 and the output layer 2. By the training function 110b, the processing circuit 110 determines these sets of coefficients by, for example, backwards propagation of errors.

By the training function 110b, the processing circuit 110 performs machine learning based on training data that is teaching data consisting of the data that is input to the input layer 1 and the data that is output to the output layer 2, determines weights each between each layer, and generates a trained model in which the weights are determined.

Note that, in deep learning, it is possible to use an auto encoder and, in this case, data necessary for machine learning need not be supervised data.

A process in the case where a trained model is applied according to the embodiment will be described. First of all, by the application function 110e, the processing circuit 110, for example, inputs an input signal to a trained model. By the application function 110e, the processing circuit 110 inputs the input signal to the input layer 1 of the neural network 7 that is the trained model. Subsequently, by the application function 110e, the processing circuit 110 acquires, as an output signal, data that is output from the input layer 2 of the neural network 7 that is the trained model. The output single is, for example, a signal on which given processing, such as noise removal, has been performed. In this manner, by the application function 110e, the processing circuit 110 generates, for example, the output signal on which the given processing, such as noise removal, has been performed. By the control function 110d, the processing circuit 110 may cause the display 135 to display the obtained output signal as required.

Back to description of the activation function and the activation layer, using FIG. 3, an activation function in the neural network 7 will be described. Nodes 10a, 10b, 10c, and 10d in FIG. 3 are displays of part of nodes of an input layer in a layer that are cut out. On the other hand, a node 11 is one of nodes of a linear layer and a node 12 is one of nodes of an activation layer that is a layer relating to the process (activation) using an activation function.

Assuming that output values of the nodes 10a, 10b, 10c and 10d are complex numbers z₁, z₂, z₃ and z₄, an output result to the node 11 in the linear layer is given by Σᵢ₌₁^{m}(ωᵢzᵢ+b), where ωᵢ is a weight coefficient between an i-th input layer and the node 11, m is the number of nodes to which the node 11 is connected, and b is a given constant. Subsequently, y representing an output result that is output to the node 12 that is an activation layer is represented by Equation (1) below using an activation function A.

$$y = A ( {\sum }_{i = 1}^{m} {ω}_{i} {x}_{i} + b ) \qquad ( 1 )$$

Here, the activation function A is generally a non-linear function and, for example, a sigmoid function, a tan h function, a ReLU (Rectified Linear Unit), or the like, is selected as the activation function A.

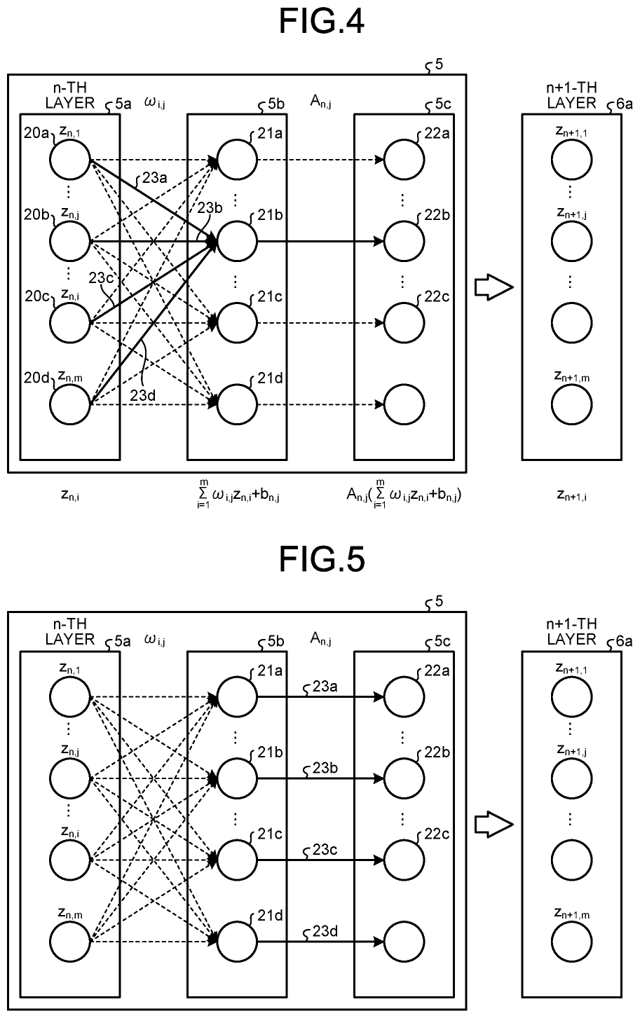

FIG. 4 illustrates the process using an activation function. The intermediate layer 5 is a n-th layer in the neural network 7 and consists of the input layer 5a, the linear layer 5b and the activation layer 5c. An input layer 5a is a n+1-th layer in the neural network. The input layer 5a includes nodes 20a, 20b, 20c, 20d, etc., the linear layer 5b includes nodes 21a, 21b, 21c, 21d, etc., and the activation layer 5c includes nodes 22a, 22b, 22c, etc. FIG. 4 illustrates a real number neural network in which each node has a real number value and an input result z_{n,1} to the input layer 5a and an output result Z_{n+1,1} to the input layer 6a are complex numbers.

A given weight addition is performed with respect to each node of the input layer 5a and accordingly an output result to the linear layer 5b is calculated. For example, an output result to the j-th node 21b in the linear layer 5b is given by Σᵢ₌₁^{m}ω_{i,j}z_{n,1}+b_{n,j}, where ω_{i,j} is a weight coefficient between the i-th input layer and the j-th linear layer and b_{n,j} is a given constant that is known as a bias term. Subsequently, the activation function A is caused to operate on each node of the linear layer 5b and accordingly output results to the activation layer 5c are calculated. For example, an output to the j-th node 22b is given by A_{n,j} (Σᵢ₌₁^{m}ω_{i,j}z_{n,i}+b_{n,j}) using an activation function A_{n,j} as presented by Equation (2) below.

$${z}_{n + 1 , j} = {A}_{n , j} ( {\sum }_{i = 1}^{m} {ω}_{i , j} {z}_{n , i} + {b}_{n , j} ) \qquad ( 2 )$$

Based on the value that is output by the nodes of the activation layer 5c, the value of each node of the input layer 6a that is an n-th layer is determined. For example, the values of the respective nodes of the activation layer 5c are directly input to the respective nodes of the input layer 6a. In another example, a further non-linear function may be caused to operate on the activation layer 5c to determine each node of the input layer 6a.

Subsequently, the background of the embodiment will be described.

In machine learning using a neural network, a real-number neural network is often used. In the field of signal processing, however, for example, a complex number expression is sometimes used in order to deal with in a unified manner two components that are an alternating current signal intensity and a time. In such a case, various applications are expected by using not a real number neural network but a complex number neural network.

As a method of dealing with a complex number in a neural network, for example, there is a method of dealing with a complex number in a neural network in which the complex number is divided into a real part and an imaginary part and each of them is considered as each node of a standard real number neural network. For example, a method of dealing with a complex number in a neural network by using a CReLU activation function that causes a ReLU to operate on each of a real part and an imaginary part of the complex number is considered.

In another example, there is a method of dealing with a complex number in a neural network by expressing the complex number using an absolute value (or an absolute value signed) and a phase and each of them is considered as each node of a standard real number neural network.

In a method of scaling the ReLU that is conventionally used in a real number neural network simply to a complex number, image quality sometimes does not increase even when training is performed because the activation function has dependence on a complex number. For example, in a method in which a complex number is simply divided into a real part and an imaginary part and a ReLU is caused to operate on each of the real part and the imaginary part, rotational symmetry about the origin that the complex number originally has is broken and a special treatment is to be made for data whose argument of complex corresponds to a 0-degree direction and a 90-degree direction compared to other directions. As a result, an artifact sometimes occurs. In this respect, while increasing the number of sets of teaching data increases accuracy of the trained model, training efficiency to the volume of data is sometimes low in the method in which an argument of complex is fixed to a specific direction and a complex number is decompose into components of the specific direction.

The data processing device 100 according to the embodiment is made in view of the above-described background and the data processing device 100 includes a processor including a complex number neural network with an activation function (CPSAF: Complex Sensitive Activation Function) sensitive to an argument of complex that is an activation function by which a gain (output) varies according to an argument of complex. Specifically, an activation function A that is used to calculate output results to the activation layers 3c, 4c and 5c of the neural network 7 that is a complex number neural network that the processing circuit 110 includes is an activation function that is sensitive to an argument of complex by which a gain varies according to the argument of complex. The gain herein means the magnitude of the output corresponding to the input. Using, for example, a plurality of activation functions sensitive to an argument of complex makes it possible to prevent an activation function from being biased with respect to components of a given direction and resultantly increase quality of an output signal.

A function A1 given by Equation (3) below is taken as a specific example of the above-described activation function (CPSFA) that is sensitive to an argument of complex.

A1_{α,β}(z)=W_{β}(phase(z)−α)z (3)

In Equation (3), z represents a complex number, phase(z) represents an argument of complex of the complex number z, and α and β represent parameters of real numbers. A gain control function W_{β}(x) is a function that is defined on a real number x and is, for example, a function for extracting an angle around x=0 by a method that is characterized by the parameter β. For example, the gain control function W_{β}(x) will be described below, taking an example of a function that has a maximum value when x=0 and whose value decreases as it separates from x=0. Note that, because angles different by constant times of 2π can be regarded as the same, for example, a periodic function with periodicity of 2π and that holds W_{β}(x+2nπ)=W_{β}(x) can be chosen, where W_{β} represents a gain control function.

In that case, an activation function A1(z) is a product obtained by multiplying the complex number z by a gain control function W_{β}(phase(z)−α) and, by the activation function A1(z), a large gain (signal value) is obtained when argument of complex of z is close to α to some extent, and the magnitude of the gain is controlled by the parameter β. Thus, an activation function A1_{α,β} that is represented by Equation (3) can be regarded as a function that is expressed by the product of the gain control function that extracts a signal component in a given angular direction and the complex number that is input and the activation function A1_{αβ} is an example of the activation function sensitive to an argument of complex.

An activation function A2(z) that is given by Equation (4) below can be taken as another example of the activation function sensitive to an argument of complex.

A2α,β(z)=A1_{α}(z) (4)

The activation function A2(z) is, in Equation (3), a special example in the case where the gain control function W_{β}(x) is given by Equation (5) below.

$${W}_{β} ( x ) = \{ \begin{matrix}1 & if \vert wrap ( x ) \vert < β \\ 0 & otherwise\end{matrix} \qquad ( 5 )$$

The warp function on the right side of Equation (5) is given by Equation (6) below, where n is a natural number.

wrap(x)=y s.t. x=2nπ+y and −π≤y<π (6)

In other words, the gain control function W_{β}(x) on the left side of Equation (5) is a function that returns 1 if the angle x is within the range of β based on 0 or returns 0 if the angle x is not within β. In other words, the activation function A2_{αβ}(z) is a function that extracts a complex number area within the range of the angle β from the direction of the angle α. In other words, the activation function A2_{αβ}(z) represented by Equation (4) can be considered as a function that extracts a signal components within the range of the given angle β from the given angle α and the activation function A2_{α,β}(z) is an example of the activation function sensitive to an argument of complex.

The embodiment of the gain control function W_{β} is not limited to the form presented by Equation (5) and the gain control function W_{β} may be, for example, in the form presented by Equation (7) or Equation (8) below.

$${W}_{β} ( x ) = \{ \begin{matrix}1 & if \vert wrap ( x ) \vert < β \\ ε & otherwise\end{matrix} \qquad ( 7 )$$

In Equation (7), for example, a small value ε=0.1 or ε=0.01 is set for c. Equation (7) is an example of a function that realizes a function that resembles to LeakyReLU with respect to an input of a complex number.

$${W}_{β} ( x ) = \{ \begin{matrix}1 & if \vert wrap ( x ) \vert < β \\ ε ( exp ( - \vert x \vert ) - 1 ) & otherwise\end{matrix} \qquad ( 8 )$$

In Equation (8), ε has a meaning similar to a minimum output value for a negative input in ELU and, for example, ε=1 is employed. Equation (8) is an example of the function that realizes a function similar to ELU with respect to a complex number input.

Activation functions A3(z) to A5(z) that are given by Equations (9) to (11) below are taken as other examples of the activation function sensitive to an argument of complex.

A3_{α,β}(z)=Re(A1_{α,β}(z)exp(−iα))exp(iα) (9)

A4_{α,β}(z)=A3_{α,β}(z)+Im(z exp(−iα))exp(iα) (10)

A5_{α,β}(z)=A_{legacy}(Re(A1_{α,β}(z)exp(−iα)))exp(iα) (11)

An activation function A3_{α,β}(z) given by Equation (9) is obtained by rotating the activation function A1_{α,β}(z) to the right by only an angle α, then taking a real part, and rotating the activation function in a direction opposite to the direction of the previous rotation operation by only the angle α. In other words, the activation function A3_{αβ}(z) is a function corresponding to an operation of rotation on the origin, an operation of taking a real part of a complex number, and an operation containing an operation of rotation in an opposite direction to the direction of the rotation operation.

An activation function A4_{α,β}(z) given by Equation (10) is obtained by rotating a complex number z to the right by only an angle α, then taking an imaginary part, and adding the complex number that is rotated in an opposite direction to the direction of the previous rotation operation by only the angle α to the activation function A3_{αβ}(z).

In Equation (11), A_{legacy} is a standard activation function that returns a real number value to a given real number value. A sigmoid function, a soft sign function, a soft plus function, a tan h function, a ReLU, a truncated power function, a polynomial, a radial basis function, and a wavelet are examples of A_{legacy}. An activation function A5_{αβ}(z) that is given by Equation (11) is basically a function similar to the activation function A3_{αβ}(z) and an operation of applying the activation function A_{legacy} that is defined by a real number after performing the operation of taking a real part is additionally contained.

Note that the activation function A1_{αβ} that is expressed using the gain control function W_{β} can be written in an expression form using a gain function G_{β} as expressed by Equation (12) below. The function G_{β} is given by Equation (13) below, where γ=cos β.

$$A {1}_{α , β}^{′} ( z ) = {G}_{β} ( z exp ( - i α ) ) exp ( i α ) z \qquad ( 12 )$$

$${G}_{β} ( γ ) = \{ \begin{matrix}1 & if - β \vert z \vert < Re ( z \cdot exp ( - i α ) ) < β \vert z \vert \\ 0 & otherwise\end{matrix} \qquad ( 13 )$$

The gain function G_{β} corresponding to the gain control function W_{β} in the form of Equation (5) has been described and, as for the gain control function W_{β} in the form of Equation (7) or Equation (8), a corresponding gain function G_{β} can be similarly constructed.

The relationship between the above-described activation functions and a Complex ReLU activation function can be described as follows: the Complex ReLU activation function is given by ReLU(Re(z))+iReLU(Im(z)) for a complex number z. While the approach to a problem differs between the activation functions A1_{αβ} to A5_{αβ} that are sensitive to an argument of complex according to the embodiment and a Complex ReLU not containing the rotation operation, the activation functions A1α_{β} to A5α_{β} that are sensitive to an argument of complex according to the embodiment contain a process close to that of the Complex ReLU (for example, α=α/4, β=π/4). Depending on the choice of a gain control function, it would be possible to perform a process equivalent to the Complex ReLU.

To perform machine learning using the neural network 7 using an activation function by which a gain varies according to an argument of complex, the processing circuit 110 according to the embodiment may apply an activation function while changing a parameter contained in the activation function. Specifically, for example, assuming that the activation function A1_{αβ} represented by Equation (3) is used, by the training function 110b, the processing circuit 110 may perform machine learning by applying the activation function A1_{αβ} to the neural network 7 while changing the angle α or β that is a parameter contained in the activation function A1_{αβ}.

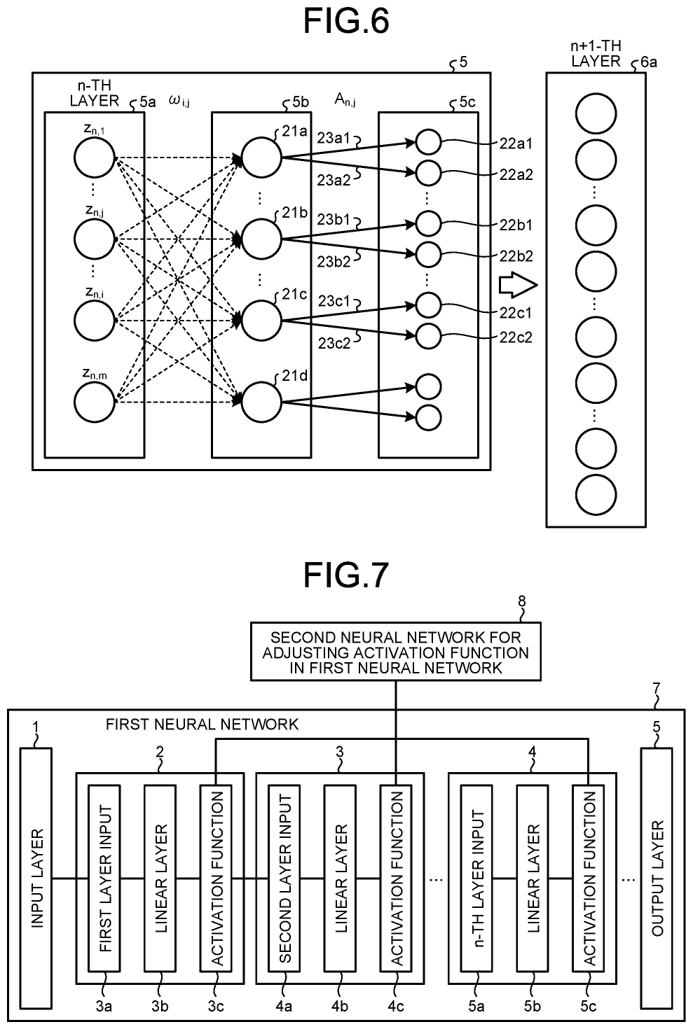

The case will be described using FIGS. 5 and 6. FIG. 5 is a diagram illustrating the case of performing training while changing a parameter contained in an activation function to different nodes. On the other hand, FIG. 6 is a diagram illustrating the case of performing training while changing a parameter contained in an activation function to the same node, that is, while applying a plurality of activation functions to a single node.

According to FIG. 5, by the training function 110b, the processing circuit 110 performs machine learning by applying an activation function by which a gain varies according to an argument of complex to the neural network 7 while changing a parameter contained in the activation function to different nodes. For example, assuming that A1_{αβ} is chosen as an activation function, by the training function 110b, the processing circuit 110 performs machine leaning by applying an activation function 23 to the neural network 7 while changing an angle α or β that is a parameter contained in the activation function A1_{αβ} to different nodes.

For example, in the example in FIG. 5, by the training function 110b, the processing circuit 110 applies an activation function A1_{αβ} where α=0 degree as the activation function 23 to the node 21a and obtains an output result to the node 22a of the activation layer 5c. By the training function 110b, the processing circuit 110 applies an activation function A1_{αβ} where α=120 degrees as the activation function 23 to the node 21b and obtains an output result to the node 22b of the activation layer 5c. By the training function 110b, the processing circuit 110 applies an activation function A1_{αβ} where α=240 degrees as the activation function 23 to the node 21c and obtains an output result to the node 22c of the activation layer 5c.

In the example described above, the parameter contained in the applied activation function A1_{αβ} applied to each node changes as represented by α=0 degree, 120 degrees or 240 degrees. Accordingly, in the complex number neural network, it is possible to make the directions of activation functions disperse and reduce adverse effects resulting from dependence of an activation function on a specific angular direction.

Some actual examples of choosing parameters α and β in an activation function will be taken. For example, by the training function 110b, the processing circuit 110 may use a fixed value β=π/4, use four types of angles α={π/4, 3π/4, 5π/4, 7π/4} as the parameter of the activation function to be changed, and perform training by applying the activation function. For example, by the training function 110b, the processing circuit 110 may fix β=π/3, use three types of angles α={π/3, π, 5π/3} as a parameter of the activation function to be changed, and perform training by applying the activation function. In another example, by the training function 110b, the processing circuit 110 may fix α=0, use three types of angles β={π/4, π/3, π/2} as a parameter of the activation function to be changed, and perform training by applying the activation function. When the parameters α and β are not changed, by the training function 110b, the processing circuit 110 may set both the values α and β to be π/3 and perform training.

By the training function 110b, the processing circuit 110 may apply an activation function while changing an amount corresponding to a certain angle that is a parameter contained in the activation function to an integral multiple of a first angle that is a value obtained by dividing 360 degrees or 180 degrees by a golden ratio. Accordingly, the activation function enables the same value almost in any angular direction, enable the values to disperse, and reduce artifacts, etc. Setting a Fibonacci value for the number of nodes in each layer of the complex number neural network enables further dispersion of values of the activation function in any direction and reduce artifacts, etc.

In the example in FIG. 5, the case of applying an activation function while changing a parameter according to the activation function to different nodes; however, embodiments are not limited to this. By the training function 110b, the processing circuit 110 may perform training by applying an activation function while changing a parameter according to the activation function to the same node. In other words, the processing circuit 110 may apply a plurality of activation functions to the same node.

FIG. 6 illustrates the example. By the training function 110b, the processing circuit 110 may perform training by applying an activation function while changing a parameter according to the activation function to the same node.

The case where an activation function is A1_{αβ} that is represented by Equation (3) as in the case illustrated in FIG. 5 will be described. By the training function 110b, the processing circuit 110 applies an activation function A1_{αβ} where α=60 degrees as an activation function 23a1 to the node 21a and obtains an output result to a node 22a1 of the activation layer 5c and applies an activation function A1_{αβ} where α=240 degrees as an activation function 23a2 to the node 21a and obtains an output result to a node 22a2 of the activation layer 5c. By the training function 110b, the processing circuit 110 applies an activation function A1_{αβ} where α=60 degrees as an activation function 23b1 to the node 21b and obtains an output result to a node 22b1 of the activation layer 5c and applies an activation function A1_{αβ} where α=240 degrees as an activation function 23b2 to a node 21b and obtains an output result to a node 22b2 of the activation layer 5c. By the training function 110b, the processing circuit 110 applies an activation function A1_{αβ} where α=60 degrees as an activation function 23c1 to the node 21c and obtains an output result to the node 22c1 of the activation layer 5c and applies an activation function A1_{αβ} where α=240 degrees as an activation function 23c2 to the node 21c and obtains an output result to a node 22c2 of the activation layer 5c.

As can been seen from the description above, in the present embodiment, complex number activation functions are applied to a node of a single linear layer and output results are multiplexed. This enables values of the activation function to disperse in directions of argument of complex and improvement in image quality.

In another example, by the training function 110b, the processing circuit 110 may apply an activation function while changing a parameter relating to the activation function according to each layer of the neural network 7. For example, by the training function 110b, using GA as a golden angle, the processing circuit 110 may apply an activation function A1_{αβ} where α=GA, α=2*GA, and α=3*GA to a first layer of the neural network 7 and apply an activation function A1_{αβ} where α=4*GA, α=5*GA, and α=6*GA to a second layer of the neural network 7. It is possible to set the parameter α of the activation function such that the same angle does not appear again over the layers of the neural network 7, that is, every angle differs. This enables the values of the activation function to disperse in a direction of an argument of complex.

For example, in the embodiment described above, the angle of the parameter α is not limited to a golden angle. For example, by the training function 110b, the processing circuit 110 may apply an activation function A1_{αβ} in which α=θ+rand(1), α=2*θ+rand(1) and α=3*θ+rand(1) where θ is an angle to the first layer of the neural network 7 and may apply an activation function A1_{αβ} in which α=4*θ+rand(2), α=5*θ+rand(2) and α=6*θ+rand(2) to the second layer of the neural network 7, where rand(i) is a random number that is determined per i and is a random number that takes a fixed value for each layer of the neural network.

The embodiment is not limited to this. The processing circuit 110 may include a calculator (not illustrated in FIG. 1) that optimizes a parameter relating to an activation function and, by the training function 110b, may perform training using an activation function based on the parameter that is optimized by the calculator and generate a trained model. FIG. 7 illustrates an example of the process.

As illustrated in FIG. 7, the processing circuit 110 includes the first neural network 7 that is a neural network that outputs an output signal/output data to an input signal/input data and a second neural network 8 for adjusting an activation function in the first neural network 7. The second neural network 8 is an example of the aforementioned calculator. The second neural network 8 is connected to the activation layers 3c, 4c and 5c of the first neural network 7 and controls a parameter of the activation function in the activation layer.

As described above, the case where an activation function A1_{αβ} is used will be described. The value of a parameter α of an activation function A1_{αβ} in the activation layers 3c, 4c and 5c of the first neural network 7 is defined as α=αᵢ+αᵢₙᵢₜ, where i denotes an i-th layer. Here, αᵢₙᵢₜ is an initial value of the parameter α, αᵢ is a correction value of the parameter α of the i-th layer and has a given value per layer. The value of αᵢ is optimized by training by the calculator.

For example, the processing circuit 110 may alternately and repeatedly execute first training that is weight coefficient training in the first neural network 7 that is executed by the training function 110b and second training that is training of the value of a parameter of an activation function of the first neural network 7 that is executed by the calculator.

In another example, after executing the second training that is training of the value of the parameter of the activation function of the first neural network 7 that is executed by the calculator, the processing circuit 110 may, using the value of the parameter, perform the first training that is training of a weight coefficient in the first neural network 7 that is executed by the training function 110b.

The processing circuit 110 may execute the first training and the second training simultaneously.

The configuration of the calculator is not limited to a neural network and, for example, the value of a parameter of an activation function of the first neural network 7 may be optimized using linear regression.

The above-described embodiment has been described as the case where a correction value of a common parameter α is used per layer and per node has been described; however, embodiments are not limited thereto, and the correction value of the common parameter α may be a common parameter per layer or may be a different parameter per layer and per node.

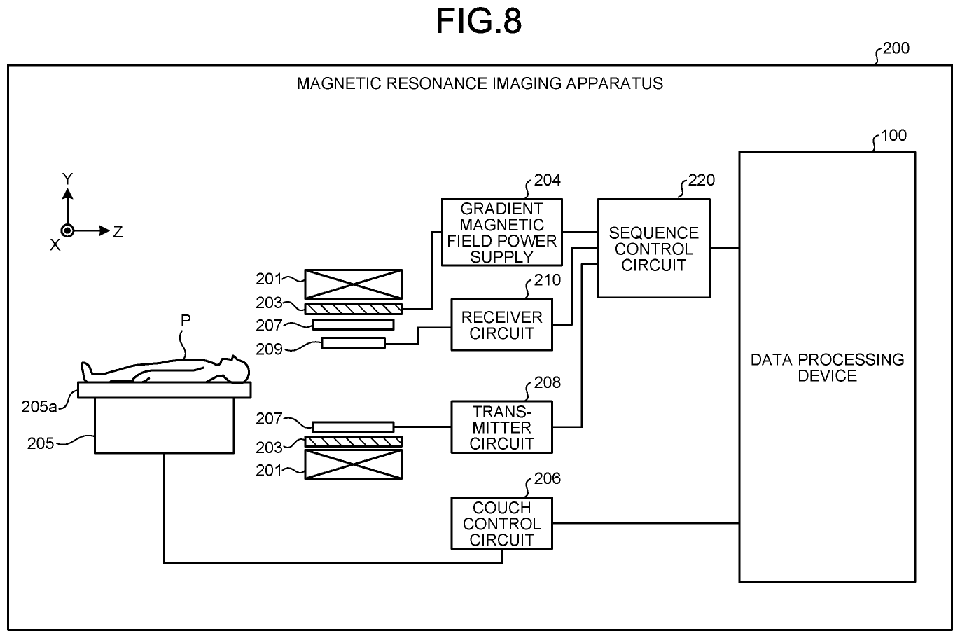

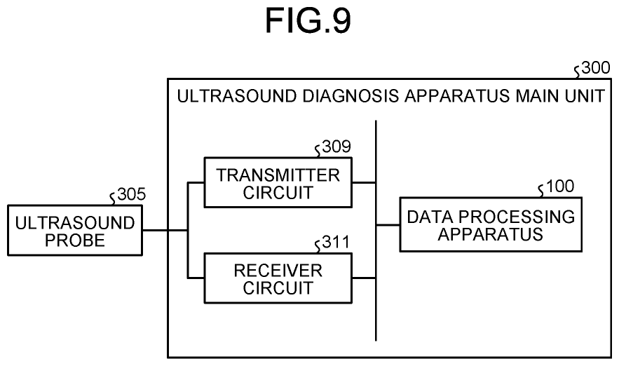

Using FIG. 8 and FIG. 9, a medical signal processing apparatus in which the data processing device 100 according to the embodiment is installed will be described as one of examples using the data processing device 100. The following description does not limit use of the data processing device 100 to the medical signal processing apparatus.

In other words, the data processing device 100 is connected to, for example, various medical image diagnosis apparatuses, such as a magnetic resonance imaging apparatus illustrated in FIG. 8 and an ultrasound diagnosis apparatus illustrated in FIG. 9, and executes processing of a signal that is received from the medical image diagnosis apparatus, generation of a trained model, execution of the trained model, etc. Note that examples of the medial image diagnosis apparatus to which the data processing device 100 is connected are not limited to the magnetic resonance imaging apparatus and the ultrasound diagnosis apparatus and the medial image diagnosis apparatus may be another device, such as an X-ray CT apparatus or a PET apparatus. For example, the data processing device 100 may be a device that processes magnetic resonance data that is not medical data.

Note that, when the processing circuit 110 is installed in various medical image diagnosis apparatuses, or when the processing circuit 110 performs processing in association with various medical image diagnosis apparatuses, the processing circuit 110 may have a function of executing processes relating to the medical image diagnosis apparatuses together.

FIG. 8 illustrates an example of a magnetic resonance imaging apparatus 200 in which the data processing device 100 according to the embodiment is installed.

As illustrated in FIG. 8, the magnetic resonance imaging apparatus 200 includes a static field magnet 201, a static magnetic field power (not illustrated in FIG. 8), a gradient coil 203, a gradient magnetic field power supply 204, a couch 205, a couch control circuit 206, a transmitter coil 207, a transmitter circuit 208, a receiver coil 209, a receiver circuit 210, a sequence control circuit 220 (sequence controller), and the data processing device 100 described using FIG. 1. The magnetic resonance imaging apparatus 200 does not include a subject P (for example, a human body). The configuration illustrated in FIG. 8 is an example only.

The static field magnet 201 is a magnet that is formed into a hollow and approximately cylindrical shape and the static field magnet 201 generates a static magnetic field in an internal space. The static field magnet 201 is, for example, a superconducting magnet and is excited in response to reception of supply of an electric current from the static magnetic field power supply. The static magnetic field power supplies an electric current to the static field magnet 201. In another example, the static field magnet 201 may be a permanent magnet and, in this case, the magnetic resonance imaging apparatus 200 need not include the static magnetic field power supply. The static magnetic field power may be included separately from the magnetic resonance imaging apparatus 200.

The gradient coil 203 is a coil that is formed into a hollow and approximately cylindrical shape and is arranged inside the static field magnet 201. The gradient coil 203 is formed by combining three coils corresponding respectively to X, Y and Z axes that are orthogonal to one another and the three coils are individually supplied with a current from the gradient magnetic field power supply 204 and generate gradient magnetic fields whose intensities vary along the respective axes X, Y and Z. The gradient magnetic fields of the respective axes X, Y and Z that are generated by the gradient coil 203 are, for example, a slice gradient magnetic field Gs, a phase encoding gradient magnetic field Ge, and a read out gradient magnetic field Gr. The gradient magnetic field power supply 204 supplies a current to the gradient coil 203.

The couch 205 includes a couch top 205a on which the subject P is laid and, under the control of the couch control circuit 206, the couch 205 inserts the couch top 205a with the subject P being laid thereon into the hollow (imaging entry) of the gradient coil 203. In general, the couch 205 is set such that its longitudinal direction is parallel to a center axis of the static field magnet 201. Under the control of the data acquisition device 100, the couch control circuit 206 drives the couch 205 to cause the couch top 205a to move in the longitudinal direction and the vertical direction.

The transmitting coil 207 is arranged inside the gradient coil 203, receives supply of an RF pulse from the transmitter circuit 208, and generates a high-frequency magnetic field. The transmitter circuit 208 supplies an RF pulse corresponding to a Larmor frequency that is determined according to the type of atom of subject and the intensity of magnetic field to the transmitting coil 207.

The receiving coil 209 is arranged inside the gradient coil 203 and receives a magnetic resonance signal (referred to as an “MR signal” as required below) that is emitted from the subject P because of the effect of the high-frequency magnetic field. On receiving the magnetic resonance signal, the receiving coil 209 outputs the received magnetic resonance signal to the receiver circuit 210.

The transmitting coil 207 and the receiving coil 209 described above are an example only and the coils may be configured by any one of or any combination of a coil with only a transmitting function, a coil with only a receiving function and a coil with a transmitting and receiving function.

The receiver circuit 210 detects the magnetic resonance signal that is output from the receiving coil 209 and generates magnetic resonance data based on the detected magnetic resonance signal. Specifically, the receiver circuit 210 generates magnetic resonance data by performing digital conversion on the magnetic resonance signal that is output from the receiving coil 209. The receiver circuit 210 transmits the generated magnetic resonance data to the sequence control circuit 220. Note that the receiver circuit 210 may be included on a gantry apparatus side including the static field magnet 201 and the gradient coil 203.

Based on sequence information, the sequence control circuit 220 drives the gradient magnetic field power supply 204, the transmitter circuit 208 and the receiver circuit 210, thereby capturing an image of the subject P. The sequence information is information that defines a procedure for performing imaging. The sequence information defines an intensity of a current to be applied by the gradient magnetic field power supply 204 to the gradient coil 203 and timing of supply of the current, an intensity of an RF pulse to be supplied by the transmitter circuit 208 to the transmitting coil 207 and timing of application of the RF pulse, timing of detection of a magnetic resonance signal by the receiver circuit 210, etc. For example, the sequence control circuit 220 is an integrated circuit, such as an application specific integrated circuit (ASIC) or a field programmable gate array (FPGA) or an electronic circuit, such as a central processing unit (CPU) or a micro processing circuit (MPU). The sequence control circuit 220 is an example of a scanning unit.

On receiving the magnetic resonance image data from the receiver circuit 210 as a result of driving the gradient magnetic field power supply 204, the transmitter circuit 208 and the receiver circuit 210 and capturing an image of the subject P, the sequence control circuit 220 transfers the received magnetic resonance data to the data processing device 100. The data processing device 100 performs entire control on the whole magnetic resonance imaging apparatus 200 in addition to the processes described using FIG. 1.

Back to FIG. 1, processes that are performed by the data processing device 100 and that are processes other than the processes described using FIG. 1 will be described. By the interface function 110c, the processing circuit 110 transmits sequence information to the sequence control circuit 220 and receives magnetic resonance data from the sequence control circuit 220. On receiving the magnetic resonance data, the processing circuit 110 including the interface function 110c stores the received magnetic resonance data in the memory 132.

By the control function 110d, the magnetic resonance data that is stored in the memory 132 is arranged in a k-space. As a result, the memory 132 stores k-space data.

The memory 132 stores the magnetic resonance data that is received by the processing circuit 110 including the interface function 110c, the k-space data that is arranged in the k-space by the processing circuit 110 including the control function 110d, image data that is generated by the processing circuit 110 including a generating function (or the application function 110e), etc.

By the control function 110d, the processing circuit 110 performs entire control on the magnetic resonance imaging apparatus 200 and controls imaging and generation of an image, display of an image, etc. For example, the processing circuit 110 including the control function 110d receives an imaging condition (such as an imaging parameter) on the GUI and generates sequence information according to the received imaging condition. The processing circuit 110 including the control function 110d transmits the generated sequence information to the sequence control circuit 220.

By the generating function not illustrated in FIG. 1 (or the application function 110e), the processing circuit 110 reads the k-space data from the memory 132 and performs reconstruction processing, such as Fourier transform, on the read k-space data, thereby generating a magnetic resonance image.

FIG. 9 is an example of a configuration of an ultrasound diagnosis apparatus 300 in which the data processing device 100 according to the embodiment is installed. The ultrasound diagnosis apparatus according to the embodiment includes an ultrasound probe 305 and an ultrasound diagnosis apparatus main unit 300. The ultrasound diagnosis apparatus main unit 300 includes a transmitter circuit 309, a receiving circuit 311 and the data processing device 100 described above.

The ultrasound probe 305 includes a plurality of piezoelectric vibrators and the piezoelectric vibrators generate ultrasound based on a drive signal that is supplied from the transmitter circuit 309 that the ultrasound diagnosis apparatus main unit 300 to be described below includes. The piezoelectric vibrators that the ultrasound probe 305 includes receive reflected waves from the subject P and converts the reflected waves into an electric signal (reflected wave signal). The ultrasound probe 305 includes a matching layer that is provided in the piezoelectric vibrators, a backing member that prevents backward propagation of ultrasound from the piezoelectric vibrators, etc. The ultrasound probe 305 is detachably connected to the ultrasound diagnosis apparatus main unit 300. The ultrasound probe 305 is an example of the scanning unit.

When ultrasound is transmitted from the ultrasound probe 305 to the subject P, the transmitted ultrasound is reflected on discontinuous plane of acoustic impedance in living tissue of the subject, is received as reflected waves by the piezoelectric vibrators that the ultrasound probe 305 includes, and the reflected waves are converted into a reflected wave signal. The amplitude of the reflected wave signal depends on a difference in acoustic impedance on the discontinuous plane on which the ultrasound is reflected. Note that, when a transmitted ultrasound pulse is reflected on a moving blood flow or a surface of the heart, or the like, because of the Doppler effect, the reflected wave signal is dependent on speed components of the mobile object with respect to the direction of transmission of ultrasound and undergoes a frequency shift.

The ultrasound diagnosis apparatus main unit 300 is a device that generates ultrasound image data based on the reflected wave signal that is received from the ultrasound probe 305. The ultrasound diagnosis apparatus main unit 300 is a device that is capable of generating two-dimensional ultrasound image data based on a two-dimensional reflected wave signal and is capable of generating three-dimensional ultrasound image data based on a three-dimensional reflected wave signal. Note that, even when an ultrasound diagnosis apparatus 300 is an apparatus dedicated to two-dimensional data, the embodiment is applicable.

As exemplified in FIG. 9, the ultrasound diagnosis apparatus 300 includes the transmitter circuit 309, the receiving circuit 311 and the data processing apparatus device 100.

The transmitter circuit 309 and the receiving circuit 311 controls transmission and reception of ultrasound that is performed by the ultrasound probe 305 based on an instruction of the data processing device 100 having a control function. The transmitter circuit 309 includes a pulse generator, a transmission delay unit, a pulser, etc., and supplies a drive signal to the ultrasound probe 305. The pulse generator repeatedly generates a rate pulse for forming transmission ultrasound at a given pulser repetition frequency (PRF). The transmission delay unit converges ultrasound that is generated from the ultrasound probe 305 into a beam and applies a delay for each piezoelectric vibrator necessary to determine transmission directivity to each rate pulse that is generated by the pulse generator. The pulser applies a drive signal (drive pulse) to the ultrasound probe 305 at timing based on the rate pulse.

In other words, the transmission delay unit changes the delay to be applied to each rate pulse, thereby freely adjusting the direction of transmission of ultrasound to be transmitted from the surface of the piezoelectric vibrator. The transmission delay unit changes the delay to be applied to each rate pulse, thereby controlling the position of point of convergence (focus of transmission) in a depth direction of transmission of ultrasound.

The receiving circuit 311 includes an amplifier circuit, an analog/digital (A/D) converter, a receiving delay circuit, an adder, and a quadrature detection circuit, performs various types of processes on the received reflected wave signal that is received from the ultrasound probe 305, and generates reception signal (reflected wave data). The amplifier circuit amplifies the reflected wave signal per channel and performs gain correction process. The A/D converter performs A/D conversion on the reflected wave signal on which gain correction has been performed. The receiving delay circuit applies a receiving delay time necessary to determine reception directivity to the digital data. The adder performs a process of addition of the reflected wave signal to which the reception delay is applied. The addition process performed by the adder enhances reflection components from the direction corresponding to the reception directivity of the reflected wave signal. The quadrature detection circuit convers the output signal of the adder into an in-phase signal (I signal) and a quadrature-phase signal (Q signal) of a baseband width. The quadrature detection circuit transmits the I signal and the Q signal (referred to as an IQ signal below) as the reception signal (reflected wave data) to the processing circuit 110. Note that the quadrature detection circuit may convert the output signal of the adder to a radio frequency (RF) signal and then transmit the RF signal to the processing circuit 110. The IQ signal and the RF signal serve as the reception signal with phase information.

To scan a two-dimensional area in the subject P, the transmitter circuit 309 causes transmission of an ultrasound beam for scanning the two-dimensional area from the ultrasound probe 305. The receiving circuit 311 generates a two-dimensional reception signal from the two-dimensional reflected wave signal that is received from the ultrasound probe 305. To scan a three-dimensional area in the subject P, the transmitter circuit 309 causes transmission of an ultrasound beam for scanning the three-dimensional area from the ultrasound probe 305. The receiving circuit 311 generates a three-dimensional reception signal from the three-dimensional reflected wave signal that is received from the ultrasound probe 305. The receiving circuit 311 generates the reception signal based on the reflected wave signal and transmits the generated reception signal to the processing circuit 110.

The transmitter circuit 309 causes the ultrasound probe 305 to transmit an ultrasound beam from a given transmitting position (transmitting scan line). The receiving circuit 311 receives a signal of reflected waves of an ultrasound beam, which is transmitted by the transmitter circuit 309, in a given receiving positon (receiving scan line) from the ultrasound probe 305. In the case where parallel simultaneous reception is not performed, the transmitting scan line and the receiving scan line are the same scan line. On the other hand, in the case where parallel simultaneous reception is performed, when the transmitter circuit 309 causes the ultrasound probe 305 to transmit an ultrasound beam once in one transmitting scan line, the receiving circuit 311 simultaneously receives signals of reflected waves originating from the ultrasound beam that the transmitter circuit 309 causes the ultrasound probe 305 to transmit as a plurality of reception beams in a plurality of given receiving positions (receiving scan lines) via the ultrasound probe 305.

The data processing device 100 is connected to the transmitter circuit 309 and the receiving circuit 311 and executes, in addition to the functions already illustrated in FIG. 1, and together with processing on the signal that is received from the receiving circuit 311 and control on the transmitter circuit 309, generation of a trained model, execution of the trained model, and various types of image processing. The processing circuit 110 includes, in addition to the functions already illustrated in FIG. 1, a B-mode processing function, a Doppler processing function, and a generating function. Back to FIG. 1, a configuration that the data processing device 100 that is installed in the ultrasound diagnosis apparatus 300 may include in addition to the configuration illustrated in FIG. 1 will be described.

Each of processing functions that are performed by the B-mode processing function, the Doppler processing function, and the generating function and a trained model are stored in a form of computer-executable programs in the memory 132. The processing circuit 110 is a processor that reads the programs from the memory 132 and executes the programs, thereby enabling the functions corresponding to the respective programs. In other words, the processing circuit 110 having read each of the programs has each of these functions.

The B-mode processing function and the Doppler processing function are an example of a B-mode processor and a Doppler processor.

The processing circuit 110 performs various types of signal processing on the reception signal that is received from the receiving circuit 311.

By the B-mode processing function, the processing circuit 110 receives data from the receiving circuit 311 and performs logarithmic amplification processing, envelope demodulation processing, logarithmic compression processing, etc., to generate data (B-mode data) in which a signal intensity is expressed by brightness.

By the Doppler processing function, the processing circuit 110 generates data (Doppler data) by performing frequency analysis on speed information from the reception signal (reflected wave data) that is received from the receiving circuit 311 and extracting mobile object information, such as a speed, dispersion and power, because of the Doppler effect, in many points.

The B-mode processing function and the Doppler processing function enable both two-dimensional reflected wave data and three-dimensional reflected wave data to be processed.

By the control function 110d, the processing circuit 110 entirely controls the processes of the ultrasound diagnosis apparatus. Specifically, the processing circuit 110 controls processes of the transmitter circuit 309, the receiving circuit 311 and the processing circuit 110 based on various setting requests that are input from the operator via the input device 134 and various control programs and various types of data that are read from the memory 132. By the control function 110d, the processing circuit 110 performs control to cause the display 135 to display ultrasound image data for display that is stored in the memory 132.

By the generating function not illustrated in the drawing, the processing circuit 110 generates ultrasound image data from data that is generated by the B-mode processing function and the Doppler processing function. By the generating function, the processing circuit 110 generates two-dimensional B-mode image data in which the intensity of reflected waves is represented by brightness from the two-dimensional B-mode data that is generated by the B-mode processing function. By the generating function, the processing circuit 110 generates two-dimensional Doppler image data presenting mobile object information from two-dimensional Doppler data that is generated by the Doppler processing function. The two-dimensional Doppler image data may be speed image data, dispersion image data, power image data or image data of a combination of these sets of image data.

By the generating function, the processing circuit 110 converts a scan line signal array of ultrasound scanning into a scan line signal array of a video format represented by television, or the like (scan conversion), thereby generating ultrasound image data for display. By the generating function, the processing circuit 110 performs, in addition to scan conversion, for example, as various types of image processing, image processing of regenerating an image with an average value of brightness (smoothing processing) using, for example, a plurality of image frames after scan conversion and image processing (edge enhancement processing) using a differential filter in the image. By the generating function, the processing circuit 110 performs various types of rendering on volume data in order to generate two-dimensional image data for displaying volume data on the display 135.

The memory 132 is also capable of storing data that is generated by the B-mode processing function and the Doppler processing function. The B-mode data and the Doppler data that are stored in the memory 132 can be called by the operator, for example, after diagnosis and the data serves as ultrasound image data for display via the processing circuit 110. The memory 132 is also capable of storing a reception signal (reflected wave data) that is output by the receiving circuit 311.

The memory 132 further stores a control program for performing transmission and reception of ultrasound, image processing, and display processing, diagnosis information (for example, a patient ID, opinions of a doctor, etc.,) and various types of data, such as a diagnosis protocol and various types of body marks, as required.

Back to FIG. 2, data that is input to the input layer 1 in FIG. 2 may be a medical image or medical image data that is acquired from a medical image diagnosis apparatus. When the medical image diagnosis apparatus is the magnetic resonance imaging apparatus 200, the data that is input to the input layer 1 is, for example, a magnetic resonance image. When the medical image diagnosis apparatus is the ultrasound diagnosis apparatus 300, the data that is input to the input layer 1 is, for example, an ultrasound image.

The input data that is input to the input layer 1 may be a medical image or various types of image data, projection data, intermediate data, or raw data before generation of a medical image. For example, when the medical image diagnosis apparatus is a PET apparatus, the input data that is input to the input layer 1 may be a PET image or various types of data before reconstruction of a PET image, for example, time-series data on coincidence counting information.

Data that is output from the output layer 2 is a medical image or medical image data and, like the data that is input to the input layer 1, the data may be various types of projection data, intermediate data, or raw data before generation of a medical image. When the purpose of the neural network 7 is denoising, for example, noise has been removed from the data that is output from the output layer 2 and thus the data is a high-quality image compared to the input image.

According to at least one of the embodiments described above, it is possible to improve image quality.

As for the above-described embodiments, the following appendants are disclosed as one aspect and selective features of the disclosure.

Note 1

A magnetic resonance imaging apparatus that is provided in one aspect of the disclosure includes a data processing device that includes a processor including a complex number neural network with an activation function by which a gain (output) changes according to an argument of complex.

Note 2

An ultrasound diagnosis apparatus that is provided in one aspect of the disclosure includes a data processing device that includes a processor including a complex number neural network with an activation function by which a gain (output) changes according to an argument of complex.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A data processing device comprising a processing circuit that includes a complex number neural network with an activation function by which an output varies according to an argument of complex.

2. The data processing device according to claim 1, wherein the activation function is a function that is expressed by a product of a gain control function that extracts a signal component in a given angular direction and a complex number that is input.

3. The data processing device according to claim 2, wherein the activation function is a function that extracts the signal component within a given angular range from the given angular direction.

4. The data processing device according to claim 1, wherein the activation function is a function corresponding to an operation of rotation on an origin, an operation of taking a real part of a complex number, and an operation of rotation in a direction opposite to that of the operation of rotation.

5. The data processing device according to claim 1, wherein the processing circuit is configured to apply the activation function while changing a parameter contained in the activation function.

6. The data processing device according to claim 5, wherein the processing circuit is configured to apply the activation function while changing the parameter to different nodes.

7. The data processing device according to claim 5, wherein the processing circuit is configured to apply the activation function while changing the parameter to the same node.

8. The data processing device according to claim 5, wherein the processing circuit is configured to apply the activation function while changing the parameter in each layer of the complex number neural network.

9. The data processing device according to claim 5, wherein the parameter is an amount corresponding to an angle and the processing circuit is configured to change the parameter to an integer multiple of a first angle.

10. The data processing device according to claim 9, wherein the first angle is a value obtained by dividing 360 degrees or 180 degrees by a golden ratio.

11. The data processing device according to claim 10, wherein the number of nodes of each layer of the complex number neural network is a Fibonacci value.

12. The data processing device according to claim 1, wherein the processing circuit is configured to
optimize a parameter relating to the activation function, and
perform training using the activation function based on the optimized parameter and generate a trained model.

13. The data processing device according to claim 1, wherein the processing circuit is configured to apply the complex number neural network to magnetic resonance data or ultrasound data.

14. A data processing method comprising generating a trained model using a complex number neural network with an activation function by which an output varies according to an argument of complex.
